(19) United States
(12) Patent Application Publication
(10) Pub. No.: US 2011/0250644 A1
(43) Pub. Date: Oct. 13, 2011

(54) FEED SUPPLEMENT FOR MAMMALIAN CELL CULTURE AND METHODS OF USE

(75) Inventors: Wai Lam W. Ling, East Brunswick, NJ (US); Anli Ouyang, Zionsville, IN (US); Matthew S. Manahan, Morristown, NJ (US)

(73) Assignee: Schering Corporation, Kenilworth, NJ (US)

(21) Appl. No.: 13/140,794

(22) PCT Filed: Dec. 17, 2009
(86) PCT No.: PCT/US09/68571

§ 371 Date: Jun. 17, 2011

Related U.S. Application Data
(60) Provisional application No. 61/139,329, filed on Dec. 19, 2008.

(51) Int. Cl. C12P 21/00; C12N 5/071

(52) U.S. Cl. 435/70.3; 435/404

(57) ABSTRACT

An improved feed supplement for culture of mammalian cells used to produce proteins is provided. The improved supplement is devoid of animal-derived components and protein hydrolysates. The invention also provides methods of using the supplement in production of a therapeutic proteins, such as an antibody. In some embodiments, the antibody is an anti-human IL-23p19 antibody.

### FIELD OF THE INVENTION

The present invention relates generally to media supplements for use in culturing cells for the production of recombinant proteins, such as antibodies.

### BACKGROUND OF THE INVENTION

Chinese hamster ovary (CHO) cell culture is frequently used to produce proteins for use as therapeutic agents, such as therapeutic antibodies. Growth medium for such cell cultures has historically included supplements of animal origin, but such supplements have recently been linked with the appearance of transmissible spongiform encephalopathies (TSEs), such as bovine spongiform encephalopathies (BSE, mad cow disease), which is linked to variant Creutzfeldt-Jakob disease (vCJD) in humans. See, e.g., Cleland et al. (2007) J. Microbiol. Meth. 69:345. In light of the resulting concern regarding such contamination, it is preferable to use production media that do not include animal-derived components for the manufacture of pharmaceutical agents.

Protein hydrolysates, such as soy hydrolysate, have also been used as supplements in cell culture medium to enhance productivity. However, the quality of such hydrolysates can vary from lot-to-lot, affecting both the quantity and quality of the product produced.

Accordingly, the need exists for improved methods for producing therapeutic proteins in CHO cells in culture that do not involve addition of animal-derived components that could introduce troublesome contaminants. Preferably, such methods would also support high-level expression of therapeutic polypeptides from CHO cells in culture.

### SUMMARY OF THE INVENTION

The present invention meets these needs and more by providing a feed supplement concentrate based on a modified 20X DMEM/F12, termed “SP feed” herein, devoid of animal components and protein lysates, and methods of using this supplement for the culture of CHO cells producing therapeutic polypeptides. In one embodiment the therapeutic polypeptide is an antibody, or antigen binding fragment thereof. In one embodiment the therapeutic polypeptide is an IgG antibody, or antigen binding fragment thereof. In some embodiments the therapeutic antibody is a chimeric, humanized or fully human antibody that specifically binds to human IL-23p19.

In one embodiment, the production feed supplement of the present invention comprises vitamin E and/or sodium selenite (Na₂SeO₄). In another embodiment, the vitamin E is present in the supplement concentrate at approximately 30.2 mg/L. In another embodiment, the sodium selenite is present in the supplement concentrate at approximately 0.3 mg/L. In a further embodiment, the production feed supplement concentrate comprises the components listed in Table 3.

In another aspect, the invention provides methods for using the supplement of the present invention to produce therapeutic proteins. In one embodiment, the method involves a forward-feeding rationale in which the amount of nutrient provided to the cell culture is based on the growth rate of the cells and nutrient consumption. In various embodiments the feed supplement of the present invention is added to a cell culture on more than one occasion during production, e.g. in as series of two or more bolus feedings, for example on days 3, 5 and 10. In another embodiment, the supplement of the present invention is added during early exponential phase, late exponential phase, and stationary phase. In various embodiments the cultures are supplemented one, two, three, four, five or more times. In still other embodiments, the supplement can be added daily, or on a continuous or semi-continuous basis, to ensure a steady concentration of components over time. In various embodiments the invention involves recovery of the protein from the culture after a suitable growth period, which recovery can be from the culture medium (supernatant), the cells (e.g. by lysis), or both.

In one embodiment, one or more components of the production feed supplement of the present invention is added to the culture separately, e.g. one, two, three or more components may be added separately from a mixture of the remaining components. In one embodiment, tyrosine and/or cysteine is added separately from the mixture of other feed components.

In another aspect, the invention provides a method of supplementing mammalian cells in culture for the production of a protein comprising adding vitamin E and/or sodium selenite to the culture medium at least once during a production run. In various embodiments, cultures are supplemented with vitamin E to a final concentration of approximately 2 mg/L, 4 mg/L or 6 mg/L. In various other embodiments, cultures are supplemented with sodium selenite to a final concentration of approximately 0.02 mg/L, 0.04 mg/L or 0.06 mg/L.

In some embodiments, the methods and feed supplement of the present invention support production of therapeutic proteins at levels (titers) at least as high as levels achieved with growth media supplemented with plant hydrolysate, such as soy hydrolysate. In various embodiments, the feed and methods of the present invention support production of a therapeutic monoclonal antibody at approximately 1.2-, 1.4-, 1.6-, 1.8- or 2.0-fold or higher titer than basal feed (glucose and glutamine only).

In some embodiments, the methods and medium of the present invention support production of therapeutic proteins that are at least as pure as is achieved with soy hydrolysate containing feed medium. In some embodiments purity is assessed after purification of an antibody by Protein-A chromatography. Purity may be determined, for example, by reverse-phase HPLC or size-exclusion chromatography (HP-SEC).

In one embodiment, the temperature of the culture is shifted during production from 37° C. to 34° C., e.g. on day 3, 4 or 5 of production.

In one embodiment the supplement of the present invention is prepared as a 20X concentrate and added to 1.33X, 2.66X and/or 4X final concentration in production runs. In other embodiments, the supplement is prepared and used as a 2X, 3X, 4X, 5X, 6X. 7X, 8X, 9X, 10X, 11X, 12X, 12X, 13X, 14X, 15X, 16X, 17X, 18X, 19X concentrate or more. In still other embodiments, the supplement is prepared and used as a 21X, 22X, 23X, 24X, 25X, 26X, 27X, 28X, 29X, 30X concentrate or more.

### BRIEF DESCRIPTION OF THE DRAWINGS

FIG. 1A shows the relative titer enhancement for three antibody production cell lines grown using commercial base media as a function of how (and if) the cultures were fed during production. The antibodies produced by the cell lines are referred to herein as antibodies A (open bar), B (hatched bar) and C (filled bar). All cells were cultured in commercial base medium with supplementation as indicated. The base media used were Sigma C5467 EX-CELL® ACF CHO medium, animal-component free, with HEPES, without L-glutamine, liquid, sterile-filtered, cell culture tested, either with aurin tricarboxylic acid (ATA) (for cells producing antibodies A and C) or without ATA (for cells producing antibody B, and antibodies D, E and F, which are discussed below). All cultures in FIGS. 1A and 1B were grown at 37° C. Cultures were supplemented with either soy hydrolysate, a commercially available feed medium concentrate, or SP feed. The commercially available feed medium was Sigma C1615 CHO Feed Bioreactor Supplement (Sigma-Aldrich, St. Louis, Mo., USA). Antibody titers were determined by reverse-phase-HPLC.

FIG. 1B shows data similar to those shown in FIG. 1A, except that data are presented for cells supplemented with both soy hydrolysate and SP feed.

FIGS. 2A-2D show the relative titer enhancement for various antibodies. FIGS. 2A and 2B show the relative titer enhancement for antibody D as a function of whether cultures were supplemented with soy hydrolysate or SP feed, in both 2L Braun bioreactors (FIG. 2A) and in shake flasks (FIG. 2B). FIG. 2C shows the relative titer enhancement in shake flasks for two clones expressing antibody E as a function of whether cultures were cultured using basal feed, supplemented with soy hydrolysate, or supplemented with SP feed. FIG. 2D shows the relative titer enhancement in shake flasks for 10 different selected clones expressing antibody F as a function of whether cultures were cultured using basal feed, supplemented with soy hydrolysate, or supplemented with SP feed. As is apparent, relative titers are normalized to titers for cultures supplemented with soy hydrolysate.

FIG. 3 shows the relative titer for an antibody production cell line grown at 37° C. (stippled bar) or 34° C. (open bar), as a function of whether the cultures were supplemented with soy hydrolysate, SP feed, or both. The cell line used in this experiment produced antibody A. Cultures grown at 37° C. had lower titers under all conditions. Data are normalized to the culture supplemented with soy hydrolysate and grown at 37° C.

FIG. 4 shows Annexin V and propidium iodide (PI) flow cytometric analyses of apoptosis in a monoclonal antibody production cell line. The cell line used in this experiment produced antibody D. The cultures were supplemented with either soy hydrolysate or SP feed. Samples were taken on days 0, 6, 13 and 19, and stained with Annexin V (x-axis) and propidium iodide (y-axis) and analyzed by flow cytometry.

FIG. 5 shows flow cytometric analyses of a monoclonal antibody production cell line (producing antibody D) supplemented with either soy hydrolysate or SP feed. On days 0, 6, 13 or 19, cells were fixed with para-formaldehyde, permeabilized and stained with fluorescein isothiocyanate (FITC)-anti-human Fc antibody. Lecoeur et. al. (1997) J. Immunol. Methods 209:111. Viable gates were set based on cell size and granularity from forward scatter/side-scatter (FSC/SSC) profiles. The histogram plots the number of cells expressing the indicated FITC intensity over a range of FITC intensities, and thus reflects the number of cells containing antibodies. The x-axis is a log scale from 0 to 10⁴ cells, and the y-axis is a linear scale of 0 to 200 counts.

### DETAILED DESCRIPTION

All references cited herein are incorporated by reference to the same extent as if each individual publication, database entry (e.g. GenBank sequences or GeneID entries), patent application, or patent, was specifically and individually indicated to be incorporated by reference. GenBank accession numbers for nucleic acid and protein sequences referenced herein refer to the contents of the database as of the filing date of this application. Although such database entries may be subsequently modified, GenBank maintains a public record of all prior versions of the sequences as a function of date, making such database entries an unambiguous reference to a specific sequence.

In addition, incorporation by reference of any patent or published patent application is intended to incorporate the sequences in the sequence listing for that patent or published patent application. For example, incorporation by reference of patents or published patent applications disclosing antibodies that specifically bind to IL-23p19 is intended to incorporate all sequences therein, including all CDRs, CDR variants, variable domains, and light and heavy chains, in both protein and nucleic acid form.

This statement of incorporation by reference is intended by Applicants, pursuant to 37 C.F.R. §1.57(b)(1), to relate to each and every individual publication, database entry (e.g. GenBank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### I. Definitions

As used herein, including the appended claims, the singular forms of words such as “a,” “an,” and “the,” include their corresponding plural references unless the context clearly dictates otherwise.

As used herein, “DMEM/F12” refers to a 1:1 mixture of Dulbecco's modified Eagle's medium (DMEM) and Ham's F12 base medium. Such medium is commercially available, for example, as Sigma EX-CELL® ACF CHO Medium (Catalog no. C5467). The feed supplement of the present invention (SP feed) is based on a modified form of 20X DMEM/F12 with reduced inorganic salts (to reduce osmolarity build-up during production), and without HEPES or phenol red. This modified form of 20X DMEM/F12 comprises components 1-46 of Table 3. Unless otherwise indicated, numbered “components” referred to herein are the components listed in Table 3. SP feed comprises all 49 components of Table 3, i.e. modified 20X DMEM/F12 with glutamine added to 15 g/L, and further supplemented with sodium selenite (0.3 mg/L) and vitamin E (30.2 mg/L), as indicated.

Unless otherwise indicated, components referred to by their number in Table 3 are used at the concentrations listed in Table 3.

For practical reasons, glutamine (component 47) is typically added to the SP feed mixture only shortly before feeding to avoid deamidation of glutamate. In addition, tyrosine (component 27) and cysteine (component 13) are not mixed with the other components ahead of time, but are instead added separately to the culture at the time of feeding. Without intending to be limited by theory, the solubility of tyrosine and cysteine precludes their addition to SP feed concentrate in advance of the feed, since they tend to fall out of solution over time. For example, for production runs involving three bolus feeds, a premixed solution of SP feed components 1-47 is added to the culture on the day of a feed by adding 6.7% of the culture volume (⅓ of the total 20% feed added over the production run). An appropriate amount of sodium selenite is added to the culture, and an appropriate amount of vitamin E is added to the culture, such that the final concentrations of all 49 components in the culture are substantially the same as they would have been if all 49 components had been added as a pre-mixed solution (concentrate) comprising the amounts provided in Table 3. Such calculations are well within the skill in the art for people who manufacture therapeutic proteins. Although component 48, component 49, and the mix of components 1-47 are added to the culture separately, they can be added in any order. The formulation provided at Table 3 can thus be viewed as a “virtual” formulation in the sense that the components of the feed concentrate may be added to the culture to achieve the same end result as if a single solution had been prepared, regardless of whether a limited number of components are added separately for practical reasons or convenience.

The supplement of the present invention, in various embodiments, encompasses compositions defined by the ratio of the components present in Table 3, regardless of the concentration at which it is formulated. Accordingly, the invention is not limited to any specific concentration. The invention encompasses the 20X concentrate form provided at Table 3, but may also encompass to any other concentration, such as less than 1X, 1X, 2X, 3X, 4X, 5X, 6X. 7X, 8X, 9X, 10X, 11X, 12X, 12X, 13X, 14X, 15X, 16X, 17X, 18X, 19X, 20X, 21X, 22X, 23X, 24X, 25X, 26X, 27X, 28X, 29X, 30X or more than 30X, and any non-integral concentration as well. It is intended that the supplement be added to give a final concentration of approximately, but not necessarily exactly, 4X.

The “X” concentrations reported herein are arbitrary, and based solely on the fact that the feed supplement of the present invention is derived from “20X” DMEM/F12 medium. Accordingly, the “X” concentrations do not reflect any specific desired working, or final, concentration. A final concentration of 4X in culture medium, for example, may be perfectly suitable. This usage may not be like typical usage, in which it is often implicit that “1X” represents some desired “final” concentration is a reaction mixture or culture medium.

As used herein, the term “antibody” may refer to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc., so long as they exhibit the desired biological activity.

As used herein, when referring to antibodies, the terms “binding fragment thereof” or “antigen binding fragment thereof” encompass a fragment or a derivative of an antibody that still substantially retains the ability to bind to its target. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its affinity for its target, e.g. no more than a 10-fold change in the dissociation equilibrium binding constant (K_{d}). Preferably, a binding fragment or derivative retains at least 25%, 50%, 60%, 70%, 80%, 90%, 95%, 99% or 100% (or more) of its binding affinity, although any binding fragment with sufficient affinity to exert the desired biological effect will be useful. It is also intended that, when specified, a binding fragment can include sequence variants with conservative amino acid substitutions that do not substantially alter its biologic activity.

An “IL-23 antagonist” is a molecule that inhibits the activity of IL-23 in any way. In some embodiments, an antibody or antigen binding fragment thereof of the present invention is an IL-23 antagonist that inhibits IL-23 signaling via the IL-23 receptor, for example by binding to a subunit of IL-23 or its receptor. In other embodiments an IL-23 antagonist is a small molecule or a polynucleotide, such as an antisense nucleic acid or siRNA.

“Interleukin-23 (or “IL-23”) means a protein consisting of two polypeptide subunits, p19 and p40. The sequence of the p19 subunit (also known as IL-23p19, IL23A) is provided at any of NCBI Protein Sequence Database Accession Numbers NP_{—}057668, AAH67511, AAH66267, AAH66268, AAH66269, AAH667512, AAH67513 or naturally occurring variants of these sequences. The sequence of the p40 subunit (also known as IL-12p40, IL12B) as described in any of NCBI Protein Sequence Database Accession Numbers NP_{—}002178, P29460, AAG32620, AAH74723, AAH67502, AAH67499, AAH67498, AAH67501 or naturally occurring variants of these sequences. All of these sequences are hereby incorporated by reference in their entireties.

“Interleukin-23R” or “IL-23R” means a single polypeptide chain consisting of the sequence of the mature form of human IL-23R as described in NCBI Protein Sequence Database Accession Number_NP 653302 (IL23R, Gene ID: 149233) or naturally occurring variants thereof. Additional IL-23R sequence variants are disclosed at WO 01/23556 and WO 02/29060. All of these sequences and documents are hereby incorporated by reference in their entireties.

“Interleukin-12Rβ1” or “IL-12Rβ1” means a single polypeptide chain consisting of the sequence of the mature form of human IL-12Rβ1 as described in NCBI Protein Sequence Database Accession Numbers NP_{—}714912, NP_{—}005526 (IL12RB1, Gene ID: 35p4) or naturally occurring variants thereof. All of these sequences and documents are hereby incorporated by reference in their entireties.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies (although these same designations, depending on the context, may also indicate the human form of a particular protein). The humanized forms of rodent antibodies will generally comprise the same CDR sequences of the parental rodent antibodies, although certain amino acid substitutions may be included to increase affinity, increase stability of the humanized antibody, or for other reasons.

Antibodies also include antibodies with modified (or blocked) Fc regions to provide altered effector functions. See, e.g., U.S. Pat. No. 5,624,821; WO 2003/086310; WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies. A longer half-life may result in less frequent dosing, with the concomitant increased convenience and decreased use of material. See Presta (2005) J. Allergy Clin. Immunol. 116:731 at 734-35.

Antibodies also include antibodies with intact Fc regions that provide full effector functions, e.g. antibodies of human isotype IgG1, which induce complement-dependent cytotoxicity (CDC) or antibody dependent cellular cytotoxicity (ADCC) in the a targeted cell. In some embodiments, the antibodies of the present invention are administered to selectively deplete cells expressing the cognate antigen from a population of cells.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding compound with a target where the association results in reduction in the normal Brownian motion of the binding compound, in cases where the binding compound can be dissolved or suspended in solution. “Binding composition” refers to a molecule, e.g. a binding compound, in combination with a stabilizer, excipient, salt, buffer, solvent, or additive, capable of binding to a target.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### II. Animal Product-Free/Hydrolysate-Free Production Feed Supplement

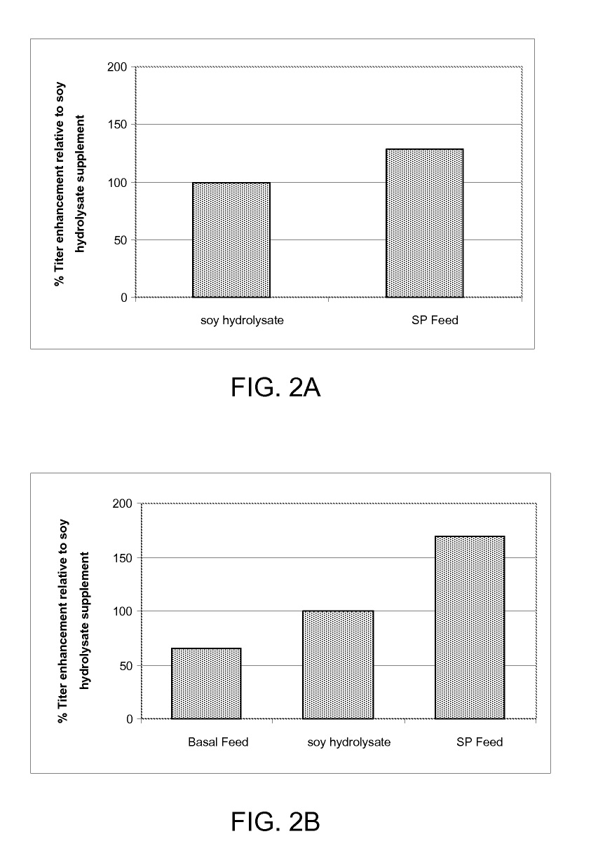

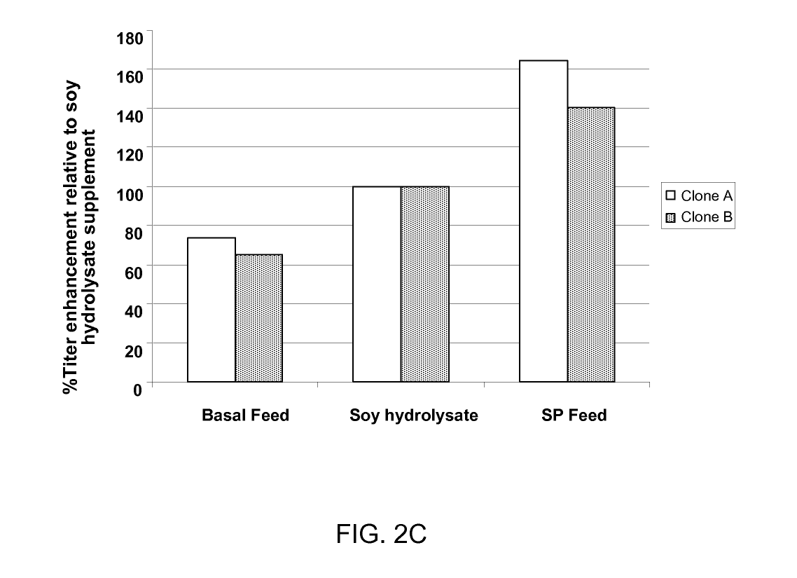

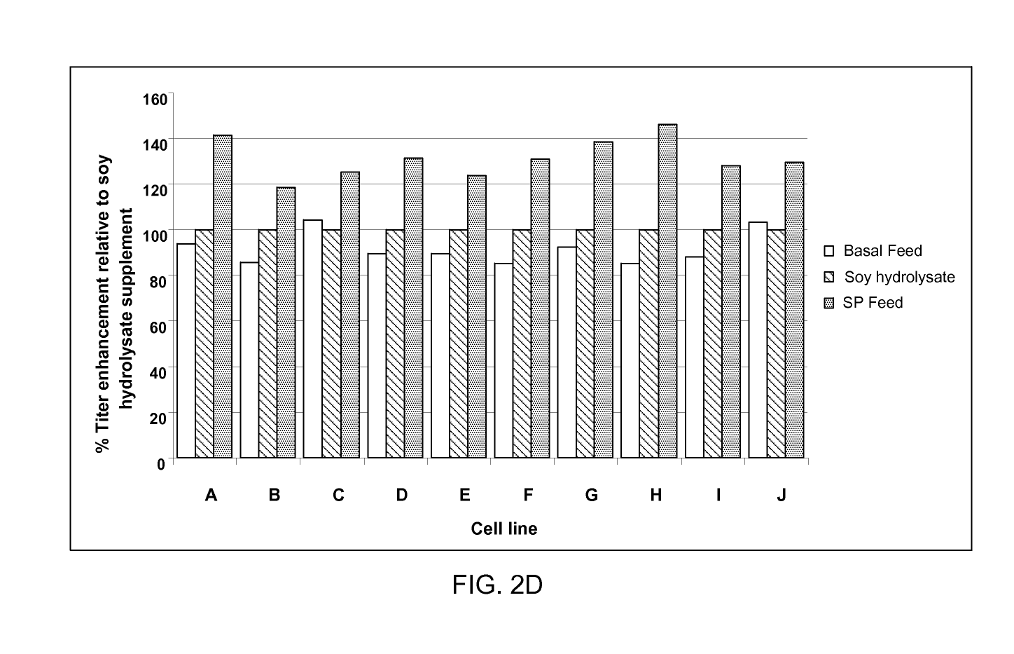

The present invention is based on a desire to eliminate reliance on animal components and poorly-defined protein hydrolysates for the production of monoclonal antibodies and protein biologics in mammalian (e.g. CHO) cell culture. The result is a chemically-defined production feed supplement with a production yield that is 25% higher than the titer of cultures supplemented with soy hydrolysate in small-scale bioreactors, and double the titer of cultures without any supplement in shake flask studies. See FIG. 2. The protein generated using this modified DMEM/F12 concentrate is of comparable purity to that produced using a hydrolysate-containing supplement.

In one embodiment, the present invention provides a high yielding monoclonal antibody (mAb) production feed supplement that is devoid of animal components and protein hydrolysates. Such supplements produce mAbs at enhanced titers and with a product quality profile that is comparable to conventional processes that use hydrolysates.

In some embodiments, the supplement may be used to grow cells to produce therapeutic antibodies, or antigen-binding fragments thereof, that specifically bind to human IL-23, for example via the p19 subunit. Exemplary antibodies that bind to human IL-23p19 are disclosed in commonly assigned Int'l Pat. Appl. Pub. No. WO 2008/103432. In other embodiments the medium may be used to produce other proteins, including antibodies that specifically bind to proteins other than IL-23p19, including antibody fragments or derivatives, cytokines, cytokine receptors, growth factors, polypeptides for use as vaccines, and even non-therapeutic proteins.

The growth medium supplement of the present invention (referred to as “SP feed”) is based on a modified, concentrated formulation of the DMEM/F12 base medium supplemented with vitamin E and sodium selenite (Na₂SeO₃). Another feeding protocol for antibody production involving supplementation with DMEM/F12 and sodium selenite has been reported. Zhou et al. (1997) Cytotechnology 24:99.

The feeding strategy is based on a forward feeding rationale, in which the amount of nutrient introduced into the cell culture is based on nutrient consumption and the growth rate of the cells. See, e.g., Zhou et al. (1997) Cytotechnology 24:99. The medium and methods of the present invention were tested in shake flasks and in small-scale stirred tank bioreactors (STR). Production process characterization and product assessment were evaluated simultaneously. Shake flasks were employed for assessing parameters, such as general characteristics of cell growth, growth as a function of temperature, effects of base medium and feed medium, and preliminary stability evaluation of cell line. In parallel, STR were used to investigate the feasibility of new production feed medium in a more controlled environment. Physiological changes that occurred with nutrient feed and process parameter changes were analyzed and monitored to reduce product deviation.

In one aspect, the invention relates to methods of culturing mammalian cells, such as CHO cells, for the production of therapeutic polypeptides, such as antibodies. Applicants studied several antibody-producing cell lines in culture to determine when cellular growth and nutrient consumption rates were the highest, using daily supplementation with SP feed. Applicants found that 1 g of glutamine was consumed for every 4 g of glucose, leading to a 1:4 ratio of glutamine to glucose in SP feed. Applicants also found that for at least some cell lines it is possible to achieve high antibody titers and production using a finite number of bolus feeds during a production run, rather than daily feeds. Feeds can be performed, for example, as three bolus feeds, for example at days 3, 5 and 10 after inoculation. The reduction in the number of feeds greatly simplifies the production run, which is of particular value in large-scale production runs, for example for preparation clinical material. Accordingly, in one embodiment, the method involves one or more bolus feeds during a production run, for example one, two, three, four, five or more bolus feedings. Such feedings are preferably performed prior to depletion of nutrients in the culture, such that cell viability and production are optimized. In some cases, such feeds can take place at days 3, 5 and 10 after inoculation.

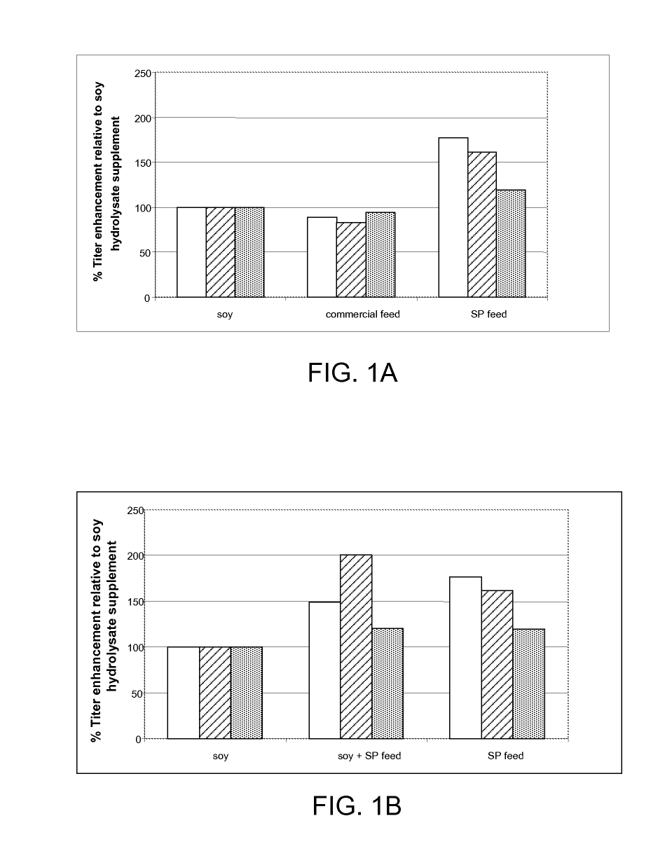

As shown in FIG. 1A, SP feed increases the final titer of antibody about 20% to 80% relative to the titer obtained when cells are supplemented with soy hydrolysate, a common additive for protein production. The feed supplement of the present invention can also be used in combination with other supplements, such as soy hydrolysate, to further enhance production by some cell lines. FIG. 1B demonstrates that while soy hydrolysate increases production by 20%, and SP feed increases production >70%, the combination increases production from the cell line producing antibody B by 90%.

As illustrated at FIGS. 2A-2D, supplementation with SP feed improved titer ˜20%-60% relative to supplementation with soy hydrolysate for a number of different antibodies. Titers were higher using SP feed in both bioreactor and shake flasks (FIGS. 2A and 2B), with titers 20-33% higher in the bioreactor with both supplements compared with shake flasks (data not shown).

Antibodies used in the experiments for which results are presented in FIGS. 1A, 1B and 2A-2D are described generally at Table 1.

TABLE 1
Antibodies Used to Assess Titer Enhancement Using SP Feed

| Antibody | Origin | Class |
|---|---|---|
| A | humanized rat | IgG1/kappa |
| B | fully human | IgG1/kappa |
| C | humanized rat | IgG1/kappa |
| D | humanized mouse | IgG1/kappa |
| E | humanized mouse | IgG1/kappa |
| F | humanized mouse | IgG4/kappa |

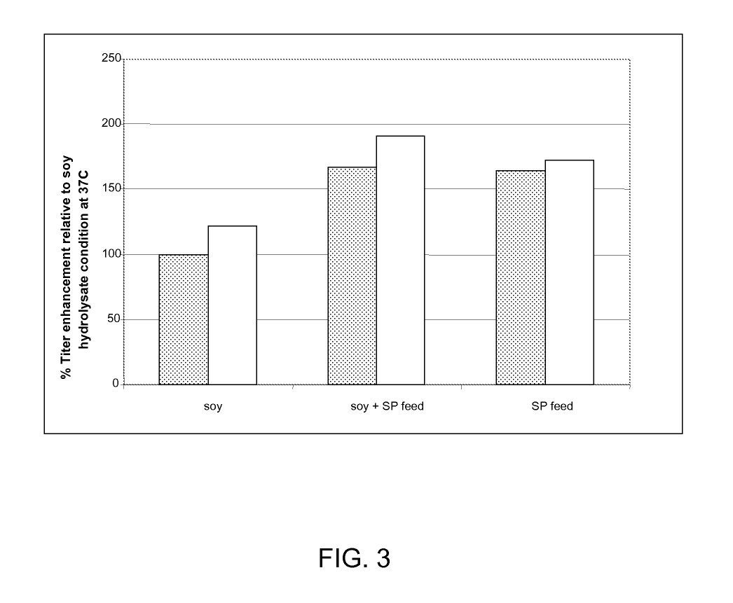

As shown in FIG. 3, cultures grown at 34° C. had higher titers than cultures grown at 37° C. regardless of the growth medium supplement. Supplementation with the SP feed improved antibody titer compared with supplementation with soy hydrolysate, and the combination of both supplements enhanced titers somewhat further.

### III. Characterization of Cells Producing Antibodies

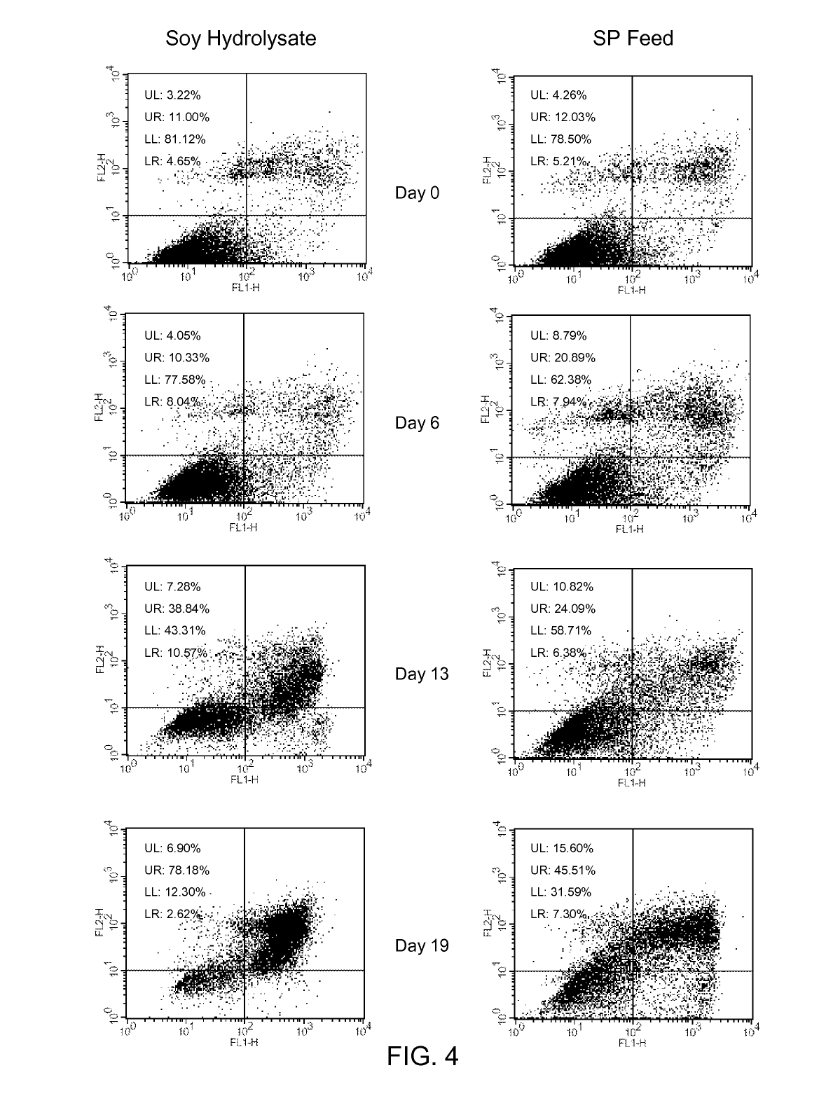

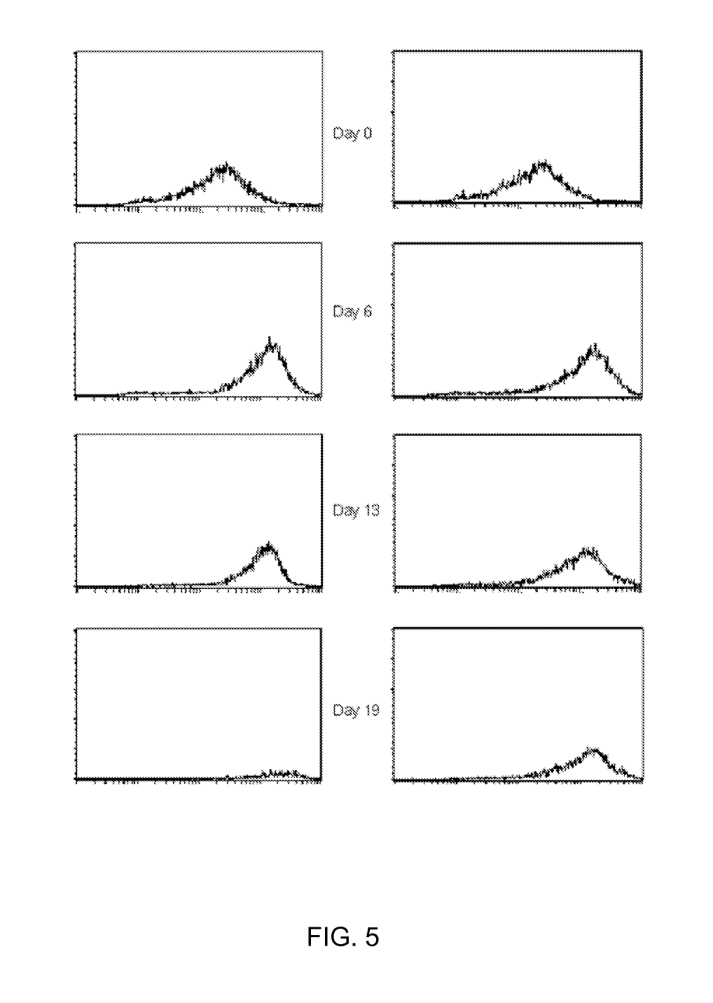

The qualities and purity of the antibody being produced may be affected by physiological changes in the producing cells. Accordingly, effects of the feed supplement of the present invention on several aspects of cellular physiology are also characterized. DNA content of the cells is measured to determine the distribution of cells within the cell cycle (data not shown), apoptotic state is determined to assess the viability of the cells (FIG. 4), and cell-associated mAb is determined to evaluate productivity (FIG. 5). All three parameters are determined by flow cytometry, e.g. using a FACSCalibur multipurpose flow cytometer system (BD Biosciences, San Jose, Calif., USA).

The cellular DNA distribution is analyzed by flow cytometry with propidium iodide staining Analysis of the percentage of cells in G0/G1, S and G2/M phases of the cell cycle shows that cultures supplemented with SP feed give a distribution in the cell cycle similar to cultures supplemented with soy hydrolysate.

The apoptotic status of the cells is analyzed by annexin V binding (using FITC-tagged annexin) and propidium iodide (PI) staining See, e.g., Vermes et. al. (1995) J. Immunol. Methods (1995) 184:39; Moore et al. (1998) Methods Cell Biol. 57:265; Tait (2008) J. Nucl. Med. 49:1573. FIG. 4 shows that on days 13 and 19 of production, a higher percentage of cells from cultures supplemented with SP feed are found in the viable gate (lower-left LL quadrant) and lower percentage are found in the late apoptotic/necrotic gate (upper-right UR quadrant) compared to cells in cultures supplemented with soy hydrolysate. Such results indicate that SP feed provides a better environment to maintain cell viability. In addition, while both feeds exhibit comparable median fluorescence intensity per cell, FIG. 5 and Table 2 (below) show that cells in cultures supplemented with SP feed show greater percentage of cell in the viable gate than the soy hydrolysate supplement condition (days 13 and 19). The net result of higher population of viable cells and comparable yield per cell means that cultures supplemented with SP feed produce significantly more antibody at Day 13, and particularly Day 19.

TABLE 2
Antibody Production and Viability

| | Soy Hydrolysate | | SP Feed | |
|---|---|---|---|---|
| | Median | | Median | |
| | Fluorescence | Cells in Viable | Fluorescence | Cells in Viable |
| Day | (relative units) | Gate (%): | (relative units) | Gate (%): |
| 0 | 213 | 99 | 199 | 99 |
| 6 | 1263 | 99 | 1346 | 97 |
| 13 | 1114 | 74 | 1084 | 94 |
| 19 | 1963 | 13 | 1333 | 71 |

### IV. Anti-IL-23 Antibodies

In general, the supplements and methods of the present invention can be used in the production of any protein from any mammalian cell line, and is particularly suited to use in production of therapeutic proteins by Chinese hamster ovary (CHO) cells in culture. In one non-limiting example, the therapeutic protein is an antibody, such as an anti-human IL-23p19 antibody (or antigen binding fragment thereof). In various embodiments, the anti-human IL-23p19 antibody comprises one, two, three, four, five or six of the CDR sequences, or the heavy and light chain variable domains, of the humanized antibodies disclosed in commonly assigned Int'l Pat. Appl. Pub. No. WO 2008/103432, the disclosure of which is hereby incorporated by reference in its entirety, for example antibody hu13B8. In another embodiment the anti-human IL-23p19 antibody competes with antibody hu13B8 for binding to human IL-23. In another embodiment the anti-human IL-23p19 antibody binds to the same epitope on human IL-23 as hu13B8. In other embodiments, the anti-human IL-23p19 antibody is able to block binding of human IL-23p19 to the antibody produced by the hybridoma deposited pursuant to the Budapest Treaty with American Type Culture Collection (ATCC—Manassas, Va., USA) on Aug. 17, 2006, under accession number PTA-7803 in a cross-blocking assay. In yet further embodiments, the anti-human IL-23p19 antibody binds to the same epitope as the antibody produced by the hybridoma deposited with ATCC under accession number PTA-7803. In still further embodiments, the anti-human IL-23p19 antibody comprises the same CDR sequences as the antibody produced by the hybridoma deposited with ATCC under accession number PTA-7803.

Additional anti-IL-23p19 antibodies suitable for production using the media and methods of the present invention are disclosed, e.g., in commonly assigned Int'l Pat. Appl. Pub. Nos. WO 2007/027714 and WO 2008/103473, the disclosures of which are hereby incorporated by reference in their entireties. Additional anti-IL-23 antibodies are disclosed, e.g., in U.S. Pat. No. 7,247,711 (to Centocor, disclosing anti-IL-23p40-specific antibodies), U.S. Pat. Appl. Pub. Nos. 2007/0009526 and 2007/0218064 (to Centocor, disclosing anti-IL-23p19 antibodies), International Pat. Appl. Pub. No. WO 2007/024846 (to Eli Lilly, disclosing anti-IL-23p19 antibodies), and International Pat. Appl. Pub. No. WO 2007/147019 (to Zymogenetics, disclosing a bispecific antibody to IL-23p19 and IL-17), the disclosures of which are hereby incorporated by reference in their entireties. Exemplary IL-12/IL-23 (anti-p40) antibodies already in clinical trials include the Centocor's fully human antibody ustekinumab (CNTO 1275) and Abbott's fully human antibody ABT-874.

In various embodiments the anti-IL-23p19 antibodies of the present invention comprise antigen binding fragments such as, but not limited to, Fab, Fab′, Fab′-SH, Fv, scFv, F(ab′)₂, and a diabody.

The broad scope of this invention is best understood with reference to the following examples, which are not intended to limit the inventions to the specific embodiments.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalog, Molecular Probes, Inc., Eugene, Oreg.; Sigma-Aldrich (2003)Catalog, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila., PA; Louis, et al. (2002) Basic Histology: Text and Atlas, McGraw-Hill, New York, N.Y.

Statistical analysis may be performed using commercially available software, including but not limited to JMP® Statistical Discovery Software, SAS Institute Inc., Cary, N.C., USA.

Cell growth media and methods are provided, e.g., at Int'l. Pat. Appl. Pub. No. WO 90/03430 and U.S. Pat. No. 5,830,761, the disclosures of which are hereby incorporated by reference in their entireties.

### Example 2

### Antibody Production

Monoclonal antibodies are produced using the feed supplements and methods of the present invention as follows. Chinese hamster ovary (CHO) cells expressing antibody D, a full-length humanized IgG anti-human IL-23p19 monoclonal antibody, are serially subcultured in vented shake flasks in base medium (BM) comprising C5467 CHO Protein-Free Medium (lacking ATA) with 1 mL/L of iron chelator C2115 (both from Sigma-Aldrich, St. Louis, Mo., USA), 20 mL/L 200 mM glutamine (Gibco, Grand Island, N.Y., USA), and 1 mL/L each of Cellgro Trace Element A and Cellgro Trace Element B (both from Mediatech, Manassas, Va., USA). Cells are incubated at 37° C. in a humidified 7.5% CO₂ incubator and shake flasks are agitated at 100 rpm on a Form a orbital shaker platform (Thermo Scientific, Waltham, Mass., USA). CHO cells are subcultured with a split ratio of 1:3 to 1:5 when viable cell density is 1-2×10⁶ cells/mL.

The effects of supplementation with SP feed on antibody (IgG) production are determined as follows. Control cultures are supplemented with soy hydrolysate by adding heat treated soy hydrolysate to a final concentration of 5 g/L using a stock solution of 200 g/L) at time zero. Glucose and glutamine are maintained above 1.5 g/L and 100 mg/L, respectively, by adding from stock solutions of glucose (450 g/L) and glutamine (200 mM).

Other cultures are supplemented with SP feed, which is a 20X concentrate, for which a recipe is provided at Table 3. SP feed is based on a modified 20X DMEM/F12 medium supplemented with sodium selenite and vitamin E (α-tocopherol), as described at Table 3 and discussed elsewhere herein. Glucose (component 46) is provided at 60 g/L and the glutamine concentration (component 47) is adjusted to 15 g/L according to pre-determined glucose to glutamine consumption ratio of 1:4.

TABLE 3
SP Feed Formulation

| Component No. | Compound | Concentration (g/L) |
|---|---|---|
| 1 | CuSO4-5H2O | 0.000026 |
| 2 | Ferric Nitrate-9H2O | 0.001 |
| 3 | Ferrous Sulfate7H2O | 0.00834 |
| 4 | Zinc Sulfate7H2O | 0.00863 |
| 5 | MgCl2 | 0.5722 |
| 6 | MgSO4 | 0.9768 |
| 7 | Sodium Phosphate monobasicH2O | 1.25 |
| 8 | Sodium phosphate Dibasic | 1.4204 |
| 9 | L-Alanine | 0.0891 |
| 10 | L-Arginine HCl | 2.9502 |
| 11 | L-Asparagine H2O | 0.15002 |
| 12 | L-Aspartic Acid | 0.133 |
| 13 | L-Cysteine HCl—H2O | 0.35122 |
| 14 | L-Cystine 2HCl | 0.62584 |
| 15 | L-Glutamic Acid | 0.14702 |
| 16 | Glycine | 0.37502 |
| 17 | L-Histidine HCl—H2O | 0.62964 |
| 18 | L-Isoleucine | 1.08948 |
| 19 | L-Leucine | 1.18108 |
| 20 | L-Lysine HCl | 1.82512 |
| 21 | L-Methionine | 0.34482 |
| 22 | L-Phenylalanine | 0.70964 |
| 23 | L-Proline | 0.34502 |
| 24 | L-Serine | 0.52504 |
| 25 | L-Threonine | 1.06908 |
| 26 | L-Tryptophan | 0.18042 |
| 27 | L-Tyrosine 2Na—2H2O | 1.11588 |
| 28 | L-Valine | 1.057 |
| 29 | d-Biotin | 0.000074 |
| 30 | D-Ca Pantothenate | 0.0448 |
| 31 | Choline Chloride | 0.1796 |
| 32 | FolicAcid | 0.053 |
| 33 | Myo-Inositol | 0.252 |
| 34 | Niacinamide | 0.04037 |
| 35 | Pyridoxal HCl | 0.04 |
| 36 | Pyridoxine HCl | 0.00062 |
| 37 | Riboflavin | 0.00438 |
| 38 | Thiamine HCl | 0.0434 |
| 39 | Vitamin B-12 | 0.0136 |
| 40 | Hypoxanthine 2NA | 0.054 |
| 41 | Linoleic Acid | 0.00084 |
| 42 | Lipoic Acid | 0.0021 |
| 43 | Putrescine 2HCl | 0.00162 |
| 44 | Sodium Pyruvate | 1.1 |
| 45 | Thymidine | 0.0073 |
| 46 | Glucose | 60 |
| 47 | Glutamine | 15 |
| 48 | Sodium Selenite | 0.0003 |
| 49 | Vitamin E | 0.0302 |

Cells are cultured in Braun bioreactors with 2L working volume (B. Braun Medical Inc., Bethlehem, Pa., USA). The inoculum is scaled up in wave bags (Wave Biotech LLC, GE Healthcare, Somerset, N.J., USA) at 37° C. and 7.5% CO₂ overlay. The bioreactors are operated at pH 6.8, dissolved oxygen (DO) of 60%, and agitation rate of 200 rpm. The temperature is initially set at 37° C. and is downshifted to 34° C. at day 3, 4 or 5. Dissolved oxygen is controlled by sparging oxygen and pH is controlled by addition of 1M NaOH or CO₂ gas.

For batches fed with SP feed, forward feeding is based on one indicator—the glucose/glutamine ratio. Feeding volume was determined by the following equations, in which Q_{glucose} is the average glucose consumption rate of 0.019 g/10⁵ cells/day, Xₙ is the viable cell density measured at Tₙ, and C_{glucose}=60 g/L.

In growth phase:

$$V = \frac{{Q}_{glucose} \cdot {\int }_{{t}_{n}}^{{t}_{n + 1}} x d t \cdot {V}_{reactor}}{{C}_{glucose}}$$

$${\int }_{{t}_{n}}^{{t}_{n + 1}} x d t ≅ \frac{( {x}_{n} + {x}_{n + 1} ) ( {t}_{n + 1} - {t}_{n} )}{2} ≅ \frac{{x}_{n} ( 1 + {e}^{μ ( {t}_{n + 1} - {t}_{n} )} ) ( {t}_{n + 1} - {t}_{n} )}{2}$$

$$μ = ln ( \frac{x}{{x}_{n - 1}} ) / ( {t}_{n} - {t}_{n - 1} )$$

In stationary phase or death phase:

$$V = \frac{{Q}_{glucose} \cdot {x}_{n} \cdot {V}_{bioreactor} \cdot ( {t}_{n + 1} - {t}_{n} )}{{C}_{glucose}}$$

Viable cell density and total cell density in shake flasks and bioreactors is measured using a Cedex automated cell culture analyzer (Innovatis AG, Bielefeld, Germany). Glucose, lactate, glutamine and glutamate are determined using a YSI 2000 analyzer (YSI, Yellow Springs Instruments Co., Ohio, USA). Ammonia is measured by Nova BioProfile 100 plus analyzer (Nova Biomedical Corp., Waltham, Mass., USA). Osmolality is measured Advanced Micro-Osmometer (Advanced Instruments, Norwood, Mass., USA). pH, pCO2, pO2 are measured by ABL5 analyzer (Radiometer America Inc., Westlake, Ohio, USA). Antibody is quantified by reverse phase HPLC or Protein-A HPLC.

Once a representative number of cultures of a given production cell line have been analyzed using the equations above to determine when feeds should be performed, and provided such cultures show sufficient reproducibility, future cultures of the same cells can simply be fed at the pre-determined times, rather than having to actively monitor the culture. For example, cultures may be fed at days 3, 5 and 10 post-inoculation. Alternatively, cultures may be fed during early exponential phase, late exponential phase, and stationary phase.

For some of the cultures studied herein, three bolus feeds of 6.67% volume each (for a total supplementation of 20% of the working volume of the culture over the course of the production run) were adequate to support high level antibody production. Accordingly, each feed comprised a dilution of the “20X” formulation of Table 3 to 1.33X final concentration in the culture medium. For non-consumable components, the second and third bolus feeds raise the concentration to 2.66X and 4X, respectively. As stated supra, the “X” concentrations reported herein are based only on the 20X DMEM/F12 medium from which the feed supplement is derived, and do not reflect any specific desired working (or final) concentration (e.g. “1X”).

Cells cultured with the addition of SP feed exhibit enhanced cell growth, reduced apoptosis at later times (e.g. days 13 and 19 post-inoculation), and give higher antibody titers than unsupplemented cultures or cultures supplemented only with soy hydrolysate. In experiments with CHO cell lines expressing antibody D, titers are up to 2-fold higher in cultures supplemented with SP feed, as compared to cultures supplemented only with soy hydrolysate.

Further experiments confirm that the antibody produced from cultures supplemented with SP feed have similar characteristics to antibody prepared using a soy hydrolysate feed when measured by reverse-phase (RP) and size-exclusion (SEC) high performance liquid chromatography (HPLC) after purification.

What is claimed is:

1. A cell feed supplement concentrate comprising components 1-47 of Table 3 and;
a) sodium selenite; or
b) vitamin E.

2. The supplement of claim 1 comprising sodium selenite and vitamin E.

3. The supplement of claim 1, wherein the sodium selenite is present at approximately 0.3 mg/L.

4. The supplement of claim 1, wherein the vitamin E is present at approximately 30.2 mg/L.

5. A method of producing a protein comprising culturing cells in growth medium supplemented with the supplement of claim 1.

6. A method of producing a protein comprising:
a) growing mammalian cells expressing the protein in culture;
b) supplementing the culture with the supplement of claim 1; and
c) recovery of the protein from the culture.

7. The method of claim 6 further comprising shifting the temperature of the culture from 37° C. to 34° C.

8. The method of claim 7 wherein the shifting of the temperature is performed on day 3, day 4 or day 5 after inoculation.

9. The method of claim 6 wherein the supplementing step (b) is repeated an additional two or more times.

10. The method of claim 10 wherein the supplementing steps are performed on days 3, 5 and 10 after inoculation.

11. The method of claim 5 or 6 wherein the cells are CHO cells.

12. The method of claim 5 or 6 wherein the protein is an antibody or antigen-binding fragment thereof.

13. The method of claim 12 wherein the antibody or antigen-binding fragment thereof comprises a human IgG constant domain.

14. The method of claim 12 wherein the antibody, or antigen-binding fragment thereof, specifically binds to human IL-23p19.

15. The method of claim 14 wherein the antibody, or antigen-binding fragment thereof, comprises at least one heavy chain CDR, and at least one light chain CDR, of antibody hu13B8b disclosed in International Pat. Appl. Pub. No. WO 2008/103432.

16. A method of producing a protein comprising:
a) growing mammalian cells expressing the protein in culture;
b) supplementing the culture with sodium selenite or vitamin E; and
c) obtaining the protein from the cell culture.

17. The method of claim 16 wherein the culture is supplemented with sodium selenite and vitamin E.

18. The method of claim 16, wherein the sodium selenite is added to the culture to give a final concentration of approximately 0.02 mg/L.

19. The method of claim 16, wherein the vitamin E is added to the culture to give a final concentration of approximately 2 mg/L.

20. The method of claim 16, wherein the sodium selenite is added to the culture to give a final concentration of approximately 0.02 mg/L and the vitamin E is added to the culture to give a final concentration of approximately 2 mg/L.
